(19) United States
(12) Patent Application Publication
(10) Pub. No.: US 2022/0270758 A1
(43) Pub. Date: Aug. 25, 2022

(54) MEDICAL INFORMATION PROCESSING APPARATUS, MEDICAL INFORMATION PROCESSING METHOD, AND NON-TRANSITORY COMPUTER-READABLE MEDIUM

(71) Applicants: CANON MEDICAL SYSTEMS CORPORATION, Tochigi (JP); Canon Kabushiki Kaisha, Tokyo (JP)

(72) Inventors: Toru TAKAHASHI, Nasushiobara (JP); Atsuko SUGIYAMA, Nasushiobara (JP); Akihiro KOGA, Shinagawa-ku (JP); Hideaki MIZOBE, Kawasaki (JP); Kohtaro UMEZAWA, Setagaya-ku (JP); Shuhei TOBA, Kawasaki (JP)

(73) Assignees: CANON MEDICAL SYSTEMS CORPORATION, Tochigi (JP); Canon Kabushiki Kaisha, Tokyo (JP)

(21) Appl. No.: 17/667,857

(22) Filed: Feb. 9, 2022

Related U.S. Application Data
(63) Continuation of application No. PCT/JP2021/007275, filed on Feb. 26, 2021.

(30) Foreign Application Priority Data
Feb. 27, 2020 (JP) 2020-031898

(51) Int. Cl. G16H 50/20; G16H 10/40; G16H 50/70

(52) U.S. Cl. CPC G16H 50/20; G16H 10/40; G16H 50/70

(57) ABSTRACT

A medical information processing apparatus according to an embodiment includes processing circuitry. The processing circuitry is configured to obtain clinical data related to a subject. The processing circuitry is configured to perform an analyzing process to analyze the obtained clinical data. The processing circuitry is configured to generate retention information for retaining the subject in a medical facility and determine at least one notification destination of the retention information, on a basis of an analysis result obtained from the analyzing process. The processing circuitry is configured to transmit the retention information to the determined notification destination.

### CROSS-REFERENCE TO RELATED APPLICATIONS

This application is a continuation of PCT international application Ser. No. PCT/JP2021/007275, filed on Feb. 26, 2021 which designates the United States, incorporated herein by reference, and which claims the benefit of priority from Japanese Patent Applications No. 2020-031898, filed on Feb. 27, 2020, incorporated herein by reference.

### FIELD

Embodiments described herein relate generally to a medical information processing apparatus, a medical information processing method, and a non-transitory computer-readable medium.

### BACKGROUND

Conventionally, when a subject has taken a medical examination (hereinafter “examination”) at a medical facility such as a hospital, the department that carried out the examination creates a report on the basis of clinical data obtained from the examination. After that, in a commonly-used workflow, the report is submitted to the primary physician so that a diagnosing process can be performed to determine a specific treatment method for the subject.

In this situation, when it is determined that the subject needs urgent treatment as a result of the diagnosing process performed by the primary physician, it is required that the treatment be provided while the subject is still in the medical facility. However, according to the abovementioned workflow, in many situations, the report is not created immediately after the examination is carried out in the department in charge of carrying out the examination. Consequently, it takes time for the subject to be diagnosed by the primary physician, and in some situations, it is difficult to provide appropriate treatment while the subject is still in the medical facility.

### BRIEF DESCRIPTION OF THE DRAWINGS

FIG. 1 is a diagram illustrating an exemplary configuration of a medical information processing apparatus according to a first embodiment;

FIG. 2 is a drawing illustrating an example of treatment execution information generated by a determining function according to the first embodiment;

FIG. 3 is a drawing illustrating another example of the treatment execution information generated by the determining function according to the first embodiment;

FIG. 4 is a flowchart illustrating a processing procedure of a process performed by processing circuitry included in the medical information processing apparatus according to the first embodiment; and

FIG. 5 is a drawing illustrating an example of a simultaneous transmission performed by a notifying function according to a third embodiment.

### DETAILED DESCRIPTION

A medical information processing apparatus according to an embodiment includes an obtaining unit, an analyzing unit, a determining unit and a notifying unit. The obtaining unit is configured to obtain clinical data related to a subject. The analyzing unit is configured to perform an analyzing process to analyze the obtained clinical data. The determining unit is configured to generate retention information for retaining the subject in a medical facility and determine at least one notification destination of the retention information, on a basis of an analysis result obtained from the analyzing process. The notifying unit is configured to transmit the retention information to the determined notification destination.

A medical information processing apparatus according to an embodiment includes a storage, an obtaining unit, an analyzing unit, a selecting unit, a determining unit and a notifying unit. The storage is configured to store a plurality of pieces of action information each defining a medical action corresponding to a state of a disease. The obtaining unit is configured to obtain clinical data related to a subject. The analyzing unit is configured to perform an analyzing process to analyze the obtained clinical data. The selecting unit is configured to select a piece of action information to be applied to the subject from among the plurality of pieces of action information stored in the storage, on a basis of an analysis result obtained from the analyzing process. The determining unit is configured to generate retention information for retaining the subject in a medical facility and determine at least one notification destination of the retention information, according to the selected piece of action information. The notifying unit configured to transmit the retention information to the determined notification destination.

Exemplary embodiments of a medical information processing apparatus will be explained in detail below, with reference to the accompanying drawings.

### First Embodiment

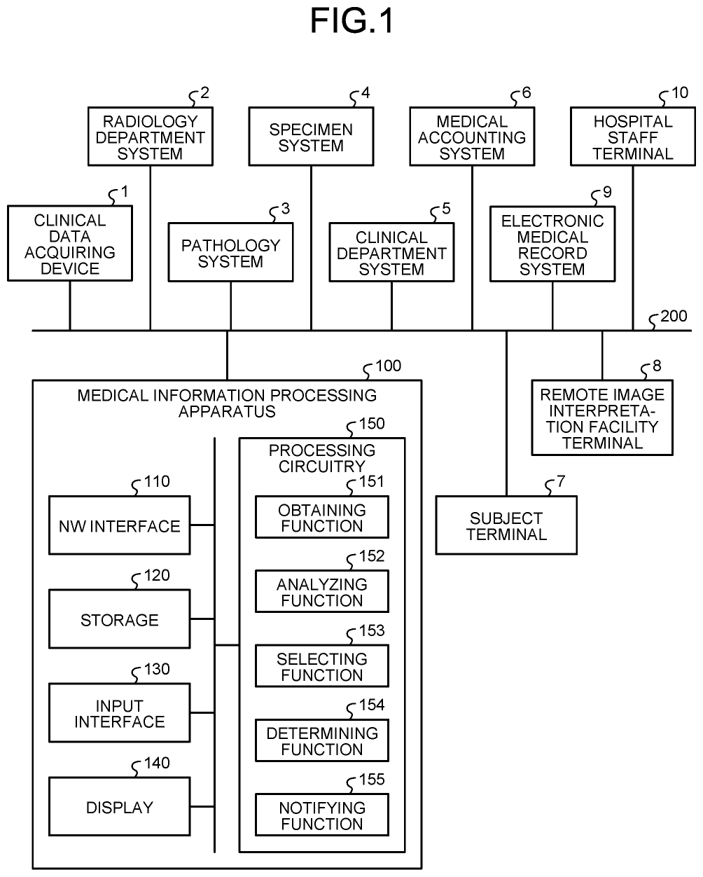

FIG. 1 is a diagram illustrating an exemplary configuration of a medical information processing apparatus according to a first embodiment.

For example, as illustrated in FIG. 1, a medical information processing apparatus 100 according to the present embodiment is communicably connected, via a network 200, to a clinical data acquiring device 1, a radiology department system 2, a pathology system 3, a specimen system 4, a clinical department system 5, a medical accounting system 6, a subject terminal 7, a remote image interpretation facility terminal 8, an electronic medical record system 9, a hospital staff terminal 10, and the like. In this situation, for example, the network 200 is realized by using a wired network, a wireless network, or a combination of the two.

In the present embodiment, among these devices and systems, the medical information processing apparatus 100, the clinical data acquiring device 1, the radiology department system 2, the pathology system 3, the specimen system 4, the clinical department system 5, the medical accounting system 6, the electronic medical record system 9, and the hospital staff terminal 10 are installed in mutually the same hospital. The hospital is an example of the medical facility.

The clinical data acquiring device 1 is a device used at the hospital in medical examinations for subjects and is a device configured to acquire clinical data related to the subjects. For example, the clinical data acquiring device 1 may be an image diagnosis apparatus used for image examinations, a Picture Archiving and Communication System (PACS), a pathological examination apparatus used for pathological examinations, a specimen examination apparatus used for specimen examinations, or the like.

In this situation, the image diagnosis apparatus is an apparatus configured to acquire image data of subjects. For example, the image diagnosis apparatus may be an X-ray Computed Tomography (CT) apparatus, a Magnetic Resonance Imaging (MRI) apparatus, an ultrasound diagnosis apparatus, an X-ray diagnosis apparatus, a Positron Emission Tomography (PET) apparatus, a Single Photon Emission Computed Tomography (SPECT) apparatus, or the like. Further, the PACS is an apparatus configured to store therein images acquired by the image diagnosis apparatus during the image examinations.

Further, the pathological examination apparatus is an apparatus configured to acquire pathological data of subjects. For example, the pathological examination apparatus is configured to acquire data related to an organ or a tissue collected from the subjects, as the pathological data. Further, the specimen examination apparatus is an apparatus configured to acquire specimen data of subjects. For example, the specimen examination apparatus is configured to acquire data related to serum or urine collected from the subjects, as the specimen data.

The radiology department system 2 is a system used in a radiology department in the hospital and is configured to manage various types of image data obtained from the image examinations using the image diagnosis apparatus. For example, the radiology department system 2 includes a terminal device used by a radiologist to reference image data when creating an image interpretation report, a terminal device used by a radiologic technologist to check a request for an image examination, a portable terminal carried around by a radiologist or a radiologic technologist, and/or the like.

The pathology system 3 is a system used in a pathological examination department in the hospital and is configured to manage various types of pathological data obtained from the pathological examinations. For example, the pathology system 3 includes a terminal device used by a pathologist to reference pathological data when creating a diagnosis report, a terminal device used by a laboratory technologist of a pathological examination to check a request for a pathological examination, a portable terminal carried around by a pathologist or a laboratory technologist, and/or the like.

The specimen system 4 is a system used in a specimen examination department in the hospital and is configured to manage various types of specimen data obtained from the specimen examinations. For example, the specimen system 4 includes a terminal device used by a laboratory technologist of a specimen examination to reference specimen data when creating an analysis report, a terminal device used by a laboratory technologist to check a request for a pathological examination, a portable terminal carried around by a laboratory technologist, and/or the like.

The clinical department system 5 is a system used in various clinical departments in the hospital and is configured to manage various types of information used when medical doctors (hereinafter “doctors”) carry out diagnosing processes and/or treatment for subjects. For example, the clinical department system 5 includes a terminal device used by a clinical department doctor to reference clinical data when carrying out a diagnosing process and/or treatment, a terminal device used by a receptionist in a clinical department to check the schedule of a diagnosis/treatment process and/or treatment, a portable terminal carried around by a clinical department doctor, a nurse, or a receptionist, and/or the like.

The medical accounting system 6 is a system used in an accounting department in the hospital and is configured to manage various types of information related to accounting. For example, the medical accounting system 6 includes a terminal device used by a receptionist in the accounting department to check subjects in and to do the accounting of diagnosis/treatment costs, a portable terminal carried around by a receptionist, an automatic payment machine used by subjects to settle the accounting of diagnosis/treatment costs, and/or the like.

The subject terminal 7 is a terminal device carried around by a subject undergoing an examination at the hospital. For example, the subject terminal 7 may be a smartphone or a tablet terminal owned by the subject himself/herself, a portable device loaned to a subject at the hospital, or the like.

The remote image interpretation facility terminal 8 is a terminal device installed at a remote image interpretation facility in a location different from the hospital in which the medical information processing apparatus 100 according to the present embodiment and the like are installed. In this situation, the remote image interpretation facility is a facility at which an image interpretation doctor works who interprets image data upon requests from other medical facilities.

The electronic medical record system 9 is a system used in the hospital and is configured to manage various types of information related to diagnosis/treatment records of subjects. For example, the electronic medical record system 9 includes a terminal device used by a doctor to input results of a question-and-answer session at the time of a diagnosing/treatment process, a terminal device used by hospital staff to reference clinical data at the time of providing medical care, and/or the like.

The hospital staff terminal 10 is a terminal device carried around by hospital staff such as a doctor, a nurse, a technologist, a receptionist, a medical accounting receptionist, or the like. For example, examples of the hospital staff terminal 10 include a smartphone or a tablet terminal owned by a doctor and a Personal Handy-phone System (PHS).

The medical information processing apparatus 100 is an information processing apparatus installed in an information management department or the like within the hospital. For example, the medical information processing apparatus 100 is realized by using a computer device such as a server, a workstation, a personal computer, or the like.

In the present example, every time an examination for a subject is carried out in the hospital, the medical information processing apparatus 100 is configured to obtain, from the clinical data acquiring device 1, clinical data acquired in the examination and to transmit the clinical data to one selected from among the radiology department system 2, the pathology system 3, and the specimen system 4.

Generally speaking, when a subject has taken am examination at a medical facility such as a hospital, a workflow is carried out in which the department that carried out the examination creates a report on the basis of clinical data obtained from the examination, so that the report is submitted to the primary physician and a diagnosing process is performed, so as to determine a specific treatment method for the subject.

For example, for an image examination using an image diagnosis apparatus (which may be referred to as “modality”) such as an X-ray Computed Tomography (CT) apparatus or a Magnetic Resonance Imaging (MRI) apparatus, an image interpretation report is created by a radiologist on the basis of image data obtained from the examination. In another example, for a pathological examination, a pathological report is created by a pathologist, on the basis of pathological data obtained from the examination. In yet another example, for a specimen examination, an analysis report is created by a laboratory technologist of the specimen examination, on the basis of specimen data obtained from the examination.

In this situation, when it is determined that the subject needs urgent treatment as a result of the diagnosing process performed by the primary physician, it is required that the treatment be provided while the subject is still in the medical facility. However, according to the abovementioned workflow, in many situations, the report is not created immediately after the examination is carried out in the department in charge of carrying out the examination. Consequently, it takes time before the primary physician performs the diagnosing process, and in some situations, it is difficult to provide appropriate treatment while the subject is still in the medical facility.

In view of the circumstances described above, in the present embodiment, the medical information processing apparatus 100 includes a configuration that makes it possible to properly provide urgent treatment for subjects.

More specifically, in the present embodiment, every time an examination for a subject is carried out in the hospital, the medical information processing apparatus 100 is configured to analyze obtained clinical data and to judge whether or not the subject needs urgent treatment. Further, when it is determined that urgent treatment is necessary, the medical information processing apparatus 100 is configured to provide a notification containing information (hereinafter “retention information”) for retaining the subject in the hospital, for an emergency doctor, the accounting department, a receptionist in the examination department, a receptionist in the clinical department, a nurse, and the subject himself/herself, and the like via the clinical data acquiring device 1, the radiology department system 2, the pathology system 3, the specimen system 4, the clinical department system 5, the medical accounting system 6, the subject terminal 7, the remote image interpretation facility terminal 8, the electronic medical record system 9, the hospital staff terminal 10, a broadcast function (a broadcast unit; not illustrated) at each facility, and/or the like; and further provides a notification containing information (hereinafter, “treatment execution information”) indicating that it is necessary to provide urgent treatment for the subject, for a radiologist, the primary physician, a clinical department doctor, an emergency doctor, a pathologist, a nurse, a laboratory technologist, and/or the like. After that, a log is saved to indicate whether or not the healthcare workers who received the notification containing the retention information have actually acknowledged the notification and whether or not the subject is actually retained.

By using this configuration, with respect to the subject who needs the urgent treatment, it is possible to prompt the relevant parties to provide treatment while the subject is still in the hospital. Consequently, according to the present embodiment, it is possible to properly provide urgent treatment for the subjects.

Next, a configuration of the medical information processing apparatus 100 described above will be explained in detail.

For example, as illustrated in FIG. 1, the medical information processing apparatus 100 includes a network (NW) interface 110, a storage 120, an input interface 130, a display 140, and processing circuitry 150.

The NW interface 110 is connected to the processing circuitry 150 and is configured to control data communication performed with other devices and apparatuses via the network 200. More specifically, under control of the processing circuitry 150, the NW interface 110 is configured to control transmission and reception of various types of data to and from the other devices and apparatuses and the systems. For example, the NW interface 110 is realized by using a network card, a network adaptor, a Network Interface Controller (NIC), or the like.

The storage 120 is connected to the processing circuitry 150 and is configured to store therein various types of data. More specifically, under control of the processing circuitry 150, the storage 120 is configured to store the various types of data therein and to also read and update the stored data. For example, the storage 120 is realized by using a semiconductor memory element such as a Random Access Memory (RAM) or a flash memory, or a hard disk, an optical disk, or the like.

The input interface 130 is connected to the processing circuitry 150 and is configured to receive input operations of various types of instructions and various types of information from an operator. More specifically, the input interface 130 is configured to convert the input operations received from the operator into electrical signals and to output the electrical signals to the processing circuitry 150. For example, the input interface 130 is realized by using a trackball, a switch button, a mouse, a keyboard, a touchpad on which an input operation can be performed by touching an operation surface thereof, a touch screen in which a display screen and a touchpad are integrally formed, a contactless input circuit using an optical sensor, an audio input circuit using a microphone, and/or the like. In the present disclosure, the input interface 130 does not necessarily have to include one or more physical operational component parts such as a mouse, a keyboard, and/or the like. Examples of the input interface 130 include, for instance, an electrical signal processing circuit configured to receive electrical signals corresponding to input operations from an external input device provided separately from the apparatus and to output the electrical signals to a controlling circuit.

The display 140 is connected to the processing circuitry 150 and is configured to display various types of information and various types of data. More specifically, under the control of the processing circuitry 150, the display 140 is configured to convert the various types of information and the various types of data into display-purpose electrical signals and to output the display-purpose electrical signals. For example, the display 140 is realized by using a Liquid Crystal Display (LCD) device, a touch panel, or the like.

In accordance with the input operations received from the operator via the input interface 130, the processing circuitry 150 is configured to control operations of the medical information processing apparatus 100. For example, the processing circuitry 150 is realized by using a processor.

Further, in the present embodiment, the storage 120 is configured, in the abovementioned structure, to store a plurality of pieces of action information each defining a medical action corresponding to a state of a disease. The storage 120 is an example of the storage.

In this situation, each of the plurality of pieces of action information is information defining an item or a procedure of urgent treatment corresponding to the state of the disease, as well as notification destinations of retention information and treatment execution information, and the like. For example, the storage 120 has stored therein action information suitable for each medical facility, in accordance with medical policies and equipment of the medical facility and statuses of doctors, nurses, staff, and the like who work at the facility.

Further, in the present embodiment, the processing circuitry 150 includes an obtaining function (an obtaining unit) 151, an analyzing function (an analyzing unit) 152, a selecting function (a selecting unit) 153, a determining function (a determining unit) 154, and a notifying function (a notifying unit) 155. The obtaining function 151 is an example of the obtaining unit. The analyzing function 152 is an example of the analyzing unit. The selecting function 153 is an example of the selecting unit. The determining function 154 is an example of the determining unit. The notifying function 155 is an example of the notifying unit.

In the processing circuitry 150, the analyzing function (the analyzing unit) 152 is configured to perform an analyzing process to analyze obtained clinical data. On the basis of an analysis result obtained from the analyzing process, the determining function (determining unit) 154 is configured to generate retention information for retaining the subject in the medical facility and to determine at least one notification destination of the retention information. The notifying function (notifying unit) 155 is configured to transmit the retention information to the determined notification destination.

Further, in the processing circuitry 150, the obtaining function (the obtaining unit) 151 is configured to obtain clinical data related to a subject. The analyzing function (the analyzing unit) 152 is configured to perform an analyzing process to analyze the obtained clinical data. On the basis of an analysis result obtained from the analyzing process, the selecting function (the selecting unit) 153 is configured to select a piece of action information to be applied to the subject, from among a plurality of pieces of action information stored in a storage. According to the selected piece of action information, the determining function (the determining unit) 154 is configured to generate retention information for retaining the subject in the medical facility and to determine at least one notification destination of the retention information. The notifying function (the notifying unit) 155 is configured to transmit the retention information to the determined notification destination.

The obtaining function 151 is configured to obtain the clinical data related to the subject. More specifically, every time an examination for a subject is carried out in the hospital, the obtaining function 151 is configured to obtain, from the clinical data acquiring device 1, clinical data acquired in the examination and to store the obtained clinical data into the storage 120.

For example, every time an image examination for a subject is carried out in the hospital, the obtaining function 151 is configured to obtain image data of the subject from either the image diagnosis apparatus or the PACS. As another example, every time a pathological examination for a subject is carried out in the hospital, the obtaining function 151 is configured to obtain pathological data of the subject from the pathological examination apparatus. As yet another example, every time a specimen examination for a subject is carried out in the hospital, the obtaining function 151 is configured to obtain specimen data of the subject from the specimen examination apparatus.

Further, the obtaining function 151 may obtain the clinical data by selecting a part of the clinical data related to the subject. Further, for example, every time an examination for a subject is carried out in the hospital, the obtaining function 151 is configured to obtain, from the clinical data acquiring device 1, clinical data selected from within examination information of the imaged site or the like and to store the obtained clinical data into the storage 120.

Further, every time an examination for a subject is carried out in the hospital, the obtaining function 151 is configured to transmit the obtained clinical data to one selected from among the radiology department system 2, the pathology system 3, and the specimen system 4.

For example, the medical information processing apparatus 100 is configured to transmit image data obtained from one of the image diagnosis apparatus and the PACS, to the radiology department system 2. As another example, the medical information processing apparatus 100 is configured to transmit pathological data obtained from the pathological examination apparatus, to the pathology system 3. As yet another example, the medical information processing apparatus 100 is configured to transmit specimen data obtained from the specimen examination apparatus to the specimen system 4.

The analyzing function 152 is configured to perform the analyzing process to analyze the clinical data obtained by the obtaining function 151.

More specifically, every time clinical data is obtained by the obtaining function 151, the analyzing function 152 is configured to read the obtained clinical data from the storage 120 and to perform the analyzing process corresponding to the type of the read clinical data.

For example, by performing an analyzing process determined in advance for each of different types of clinical data, the analyzing function 152 is configured to detect a specific disease from the clinical data.

On the basis of an analysis result obtained from the analyzing process performed by the analyzing function 152, the selecting function 153 is configured to select a piece of action information to be applied to the subject, from among the plurality of pieces of action information stored in the storage 120.

More specifically, on the basis of the analysis result obtained from the analyzing process, the selecting function 153 is configured to judge whether or not the subject needs urgent treatment. When it is determined that urgent treatment is necessary, the selecting function 153 is configured to select the piece of action information to be applied to the subject.

For example, on the basis of the analysis result, the selecting function 153 is configured to calculate a degree of urgency corresponding to the state of the disease of the subject and, when the calculated degree of urgency is higher than a threshold value, the selecting function 153 is configured to determine that urgent treatment is necessary. For example, as the degree of urgency, the selecting function 153 calculates a percentage indicating the possibility of being a disease that requires urgent treatment (e.g., a 70% possibility of being urgent pneumothorax). When the calculated percentage is higher than the threshold value, the selecting function 153 determines that urgent treatment is necessary.

According to the piece of action information selected by the selecting function 153, the determining function 154 is configured to generate the retention information for retaining, in the hospital, the subject determined to be requiring the urgent treatment and to determine at least one notification destination of the retention information.

In this situation, there are various examples of the retention information generated by the determining function 154. For example, as the retention information, the determining function 154 may generate information instructing a healthcare worker to retain the subject in the hospital. As another example, as the retention information, the determining function 154 may generate information that guides the subject so as to be able to undergo the urgent treatment.

Also, there are various examples of the notification destination of the retention information determined by the determining function 154. For example, the determining function 154 may determine the notification destination of the retention information in accordance with the requester that requested the clinical data. As another example, the determining function 154 may obtain the current position of the subject and determine the notification destination of the retention information in accordance with the current position. As yet another example, the determining function 154 may determine the notification destination of the retention information in accordance with medical information, personal information, and/or the like of the subject.

In a specific example, the determining function 154 is configured to generate retention information instructing that the subject be retained in the hospital and to determine a receptionist in the accounting department as a notification destination of the retention information. In this situation, for example, the determining function 154 may arrange the retention information to contain information that controls the medical accounting system 6 so as not to do the accounting for the subject. In addition, the determining function 154 may further determine a receptionist, the primary physician, and/or a nurse in the clinical department in which the primary physician of the subject works, as notification destinations. Alternatively, the determining function 154 may at first determine the primary physician as a notification destination, and when the primary physician determines that a receptionist also needs to be notified, the determining function 154 may further determine the receptionist in the clinical department as another notification destination.

In yet another example, the determining function 154 may generate the retention information instructing that the subject be retained in the hospital, and when the clinical data of the subject had been requested by a general clinical department, the determining function 154 may determine a receptionist, the primary physician, and/or a nurse in the clinical department as notification destinations. In contrast, when the clinical data of the subject had been requested by the emergency department, the determining function 154 may determine an emergency doctor as a notification destination. In those situations, for example, the determining function 154 identifies the department that had requested the clinical data, by referring to order information managed by an ordering system such as a Hospital Information System (HIS) used at the hospital.

In yet another example, the determining function 154 may generate the retention information instructing that the subject be retained, and when the clinical data of the subject had been requested by a general clinical department, the determining function 154 may determine a receptionist, the primary physician, and/or a nurse in the clinical department as notification destinations. In contrast, when the clinical data of the subject had been requested by the emergency department, the determining function 154 may determine an emergency doctor as a notification destination. In those situations, for example, the determining function 154 identifies the department that had requested the clinical data, by referring to order information managed by an ordering system such as a Hospital Information System (HIS) used at the hospital.

In yet another example, the determining function 154 may generate retention information that guides the subject to go to the radiology department for an additional image examination and determine the subject himself/herself as a notification destination of the retention information. In this situation, for example, the determining function 154 may arrange the retention information to contain information indicating the location of the radiology department, or the like. Further, the determining function 154 may identify the position of the subject by using cameras installed in various places within the hospital, a Global Positioning System (GPS), or the like, so as to determine a receptionist in the accounting department or a clinical department as a notification destination when the subject is inside the hospital and to determine the subject himself/herself as a notification destination when the subject is not inside the hospital.

In yet another example, the determining function 154 may select a plurality of relevant parties as candidates for the notification destinations of the retention information instructing that the subject be retained in the hospital, so as to check attendance statuses of the relevant parties by using a work attendance management system or the like in the hospital and to determine only the attending relevant parties as notification destinations. In that situation, for example, when there are two or more attending relevant parties, the determining function 154 may assess priority ranking of the relevant parties so as to determine only the relevant party in the highest priority rank as a notification destination. In yet another example, the determining function 154 may determine information indicating that the subject is not retained, as the retention information.

In yet another example, at the time of providing a notification containing the retention information or the treatment execution information, the determining function 154 may be configured to obtain, from any of the various types of systems such as the electronic medical record system 9, subject information indicating up to which process in a workflow has been completed, the workflow starting with the abovementioned subject taking the examination and ending with a specific treatment method for the subject being determined. After that, the determining function 154 may determine one or more notification destinations to be provided with a notification containing the retention information, on the basis of the obtained subject information. For example, at the time of providing the notification containing the retention information, when it is determined on the basis of the subject information obtained from the electronic medical record system 9 that the subject has completed the workflow up to a process (e.g., an image examination) preceding the accounting process, the determining function 154 may determine a person in charge of the accounting as a notification destination and may further generate retention information indicating that the subject has completed the workflow up to the process preceding the accounting process and determine a relevant party such as a nurse as a notification destination.

In yet another example, at the time of determining a notification destination of the retention information, the determining function 154 may obtain the subject information from the electronic medical record system 9 and, when it is determined on the basis of the obtained subject information that the subject is currently undergoing emergency care, the determining function 154 may determine an emergency doctor as a notification destination of the retention information. In yet another example, at the time of determining a notification destination of the retention information, the determining function 154 may obtain the subject information from the electronic medical record system 9, and when it is determined on the basis of the obtained subject information that the subject completed the accounting flow and has already left the hospital, for example, the determining function 154 may determine a nurse or the like as a notification destination of retention information containing a mobile phone number, the address, and/or the like to be used for contacting the subject. In other words, the notification destination of the retention information varies depending on whether the time (timing) at (with) which the retention information was obtained was before a specific event (e.g., the accounting process) or after the specific event (e.g., the accounting process). The determining function 154 is configured to acquire the time at which the retention information was obtained and to determine at least one notification destination of the retention information, in accordance with whether the time at which the retention information was obtained was before the specific event or after the specific event.

Further, according to the piece of action information selected by the selecting function 153, the determining function 154 is configured to generate the treatment execution information for notifying that it is necessary to provide urgent treatment for the subject, and to determine at least one notification destination of the treatment execution information.

In this situation, there are various examples of the treatment execution information generated by the determining function 154. For example, as the treatment execution information, the determining function 154 may generate information instructing a healthcare worker about an item or a procedure of the urgent treatment. In another example, as the treatment execution information, the determining function 154 may generate information indicating a degree of urgency of the urgent treatment for the subject.

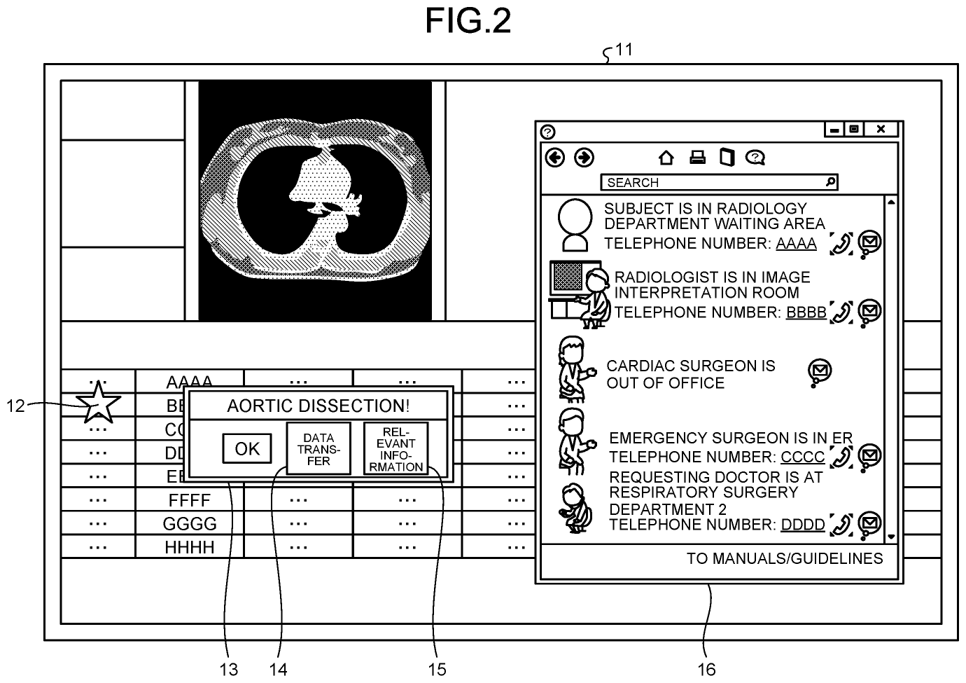

FIG. 2 is a drawing illustrating an example of the treatment execution information generated by the determining function 154 according to the first embodiment.

For example, as illustrated in FIG. 2, the determining function 154 is configured to generate treatment execution information for displaying information necessary to provide the urgent treatment, as an icon 12, a menu 13, and a window 16 on a display 11 of a terminal device used by a radiologic technologist for referencing image data. Accordingly, for example, from within the list containing pieces of image data displayed on the display 11, a piece of image data that needs to be interpreted urgently is displayed with the icon 12 of a star symbol. Further, for example, the display 11 displays: information indicating that an aortic dissection has been detected by analyzing the image data; a button 14 used for transferring the image data to a clinical department doctor; and the menu 13 arranged with a button 15 used for displaying relevant information and the like. Further, for example, the display 11 displays the window 16 listing whereabouts and contact information of relevant parties of the urgent treatment.

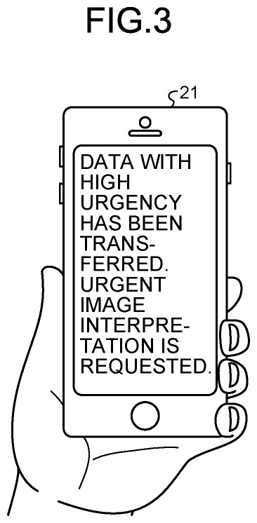

FIG. 3 is a drawing illustrating another example of the treatment execution information generated by the determining function 154 according to the first embodiment.

For example, as illustrated in FIG. 3, the determining function 154 may generate treatment execution information for displaying information necessary to provide the urgent treatment, on a display of a portable terminal 21 used by a clinical department doctor. Accordingly, for example, the display of the portable terminal 21 displays information indicating that images need to be interpreted urgently and a message including information indicating a degree of urgency (e.g., “WITH HIGH URGENCY”). In this situation, for example, when the selecting function 153 has calculated a percentage indicating the possibility of being a disease that requires urgent treatment, as the degree of urgency, the percentage may be displayed.

Further, there are various examples of the notification destination of the treatment execution information determined by the determining function 154. For example, the determining function 154 may determine the notification destination of the treatment execution information in accordance with the requester that requested the clinical data. In another example, the determining function 154 may check whereabouts of a healthcare worker involved in the urgent treatment, so as to determine the notification destination of the treatment execution information in accordance with the whereabouts. In yet another example, the determining function 154 may determine the notification destination of the treatment execution information in accordance with medical information, personal information, and/or the like of the subject.

In a specific example, the determining function 154 may generate treatment execution information instructing that an image examination be carried out again to provide the urgent treatment and may determine a radiologist as a notification destination of the treatment execution information. In this situation, for example, the determining function 154 may arrange the treatment execution information to contain information for registering a new piece of order information into the radiology department system 2. As yet another example, the determining function 154 may arrange the treatment execution information to contain information indicating whether a contrast agent is required or not, the type of the image data, an image taking condition, and/or the like.

In yet another example, when a liver disease is detected by analyzing image data of the abdomen of the subject, the determining function 154 may generate treatment execution information indicating that a diagnosing process needs to be urgently performed on the abdomen and may determine a clinical doctor for the internal organs as a notification destination of the treatment execution information. In contrast, when a lung disease is detected, the determining function 154 may generate treatment execution information indicating that a diagnosing process needs to be urgently performed on the lungs and may determine a clinical doctor for the respiratory system as a notification destination of the treatment execution information. Further, for example, when urgent treatment is necessary regarding a disease outside the specialty of the primary physician who requested the examination, the determining function 154 may provide the primary physician with a notification containing treatment execution information having added thereto information about a clinical doctor (a specialist) capable of addressing the treatment. In that situation, instead of directly requesting the clinical doctor to perform the urgent treatment, it is possible to make the request through the primary physician.

In yet another example, the determining function 154 may check to see whether or not it is possible to perform a diagnosing process at the hospital on the disease detected by analyzing the clinical data and may generate treatment execution information instructing that the subject be transferred to another hospital when image interpretation is impossible and further determine a nurse in the clinical department and staff who can arrange the hospital transfer as notification destinations of the treatment execution information. In this situation, for example, the determining function 154 may arrange the treatment execution information to contain information indicating the other hospital serving as the transfer destination, information indicating a transport method (e.g., an ambulance, a taxi, or a helicopter) for the subject, and the like. Further, the determining function 154 may at first determine the primary physician as a notification destination so that, when the primary physician determines that the subject needs to be transferred to another hospital, a nurse in the clinical department and staff who can arrange the hospital transfer are further determined as notification destinations.

In yet another example, the determining function 154 may check to see whether or not it is possible to interpret image data at the hospital by analyzing the image data and, when the image interpretation is impossible, the determining function 154 may obtain information about a remote image interpretation facility capable of addressing the situation urgently. After that, the determining function 154 may generate treatment execution information requesting the urgent image interpretation and determine the discovered remote image interpretation facility as a notification destination of the treatment execution information. In that situation, the determining function 154 arranges the treatment execution information to contain the image data of which interpretation is requested.

In yet another example, the determining function 154 may obtain information indicating the health condition of the subject and generate treatment execution information of which the content corresponds to the obtained health condition. In this situation, for example, as the information indicating the health condition of the subject, the determining function 154 may obtain information such as blood pressure, the number of walking steps, sleeping hours, and/or the like from Personal Health Records (PHR) of the subject recorded in a smartphone, a wearable terminal, or the like owned by the subject or may obtain the subject's body temperature, blood pressure, and/or the like from Electric Health Records (EHR) of the subject saved in an electronic medical record system or the like.

In yet another example, when the determining function 154 determines a plurality of notification destinations based on a sequential order determined in advance as the notification destinations of the retention information or the treatment execution information, the notifying function 155 may, for instance, transmit the retention information and the treatment execution information to the determined notification destinations in the determined sequential order. On such occasion, the notifying function 155 may be configured so that a healthcare worker included in the sequential order of transmission is able to cancel the ongoing transmission, to add another notification destination, or to change the sequential order of transmission at his/her own discretion. In that situation, the notifying function 155 may at first present one or more healthcare workers with the sequential order of transmission. As an example of the sequential order determined in advance, the determining function 154 determines to transmit information, in a medical emergency, to an emergency doctor first and to a nurse and a receptionist in the accounting department secondly. After that, the notifying function 155 at first transmits the information to the emergency doctor and subsequently receives an instruction from the emergency doctor indicating whether the transmission to the healthcare workers thereafter should be cancelled for the reason that, for example, the subject is still with the emergency doctor. In another example, the determining function 154 determines to transmit information, in a diagnosis/treatment process, to a radiologist first and to a nurse and a receptionist in the accounting department secondly. The notifying function 155 at first transmits the information to the radiologist and subsequently receives an instruction from the radiologist indicating whether the transmission to the nurse and the receptionist in the accounting department should be carried out. Further, to have the subject information checked by another doctor (e.g., a brain surgeon) at an early stage, the notifying function 155 may receive an instruction from the radiologist indicating whether treatment execution information should be transmitted to the additional notification destination.

As for the formats of the retention information and the treatment execution information generated by the determining function 154, for example, the information may be displayed in a format using an icon, a menu, a window, and the like rendered on a display as illustrated in FIGS. 2 and 3 or in a format using an electronic mail (E-mail), a short message, or the like. Further, possible formats are not limited to these examples, and it is also acceptable to adopt an audio format using speech synthesis or the like. In that situation, the notifying function 155 may provide a notification containing information using the audio format through a public address system at the hospital and/or to a smartphone carried around by the hospital staff, a smartphone of the subject, or the like.

Returning to the description of FIG. 1, the notifying function 155 is configured to transmit the retention information to the one or more notification destinations determined by the determining function 154. Further, the notifying function 155 is configured to transmit the treatment execution information to the one or more notification destinations determined by the determining function 154.

For example, as for the information of which the notification destination has been determined as a radiologist or a radiologic technologist, the notifying function 155 transmits the information to the radiology department system 2 so as to cause the information to be displayed on a terminal device, a portable terminal, or the like used by each person. In another example, as for the information of which the notification destination has been determined as a pathologist or a laboratory technologist of pathological examinations, the notifying function 155 transmits the information to the pathology system 3 so as to cause the information to be displayed on a terminal device, a portable terminal, or the like used by each person.

In yet another example, as for the information of which the notification destination has been determined as a laboratory technologist of specimen examinations, the notifying function 155 transmits the information to the specimen system 4 so as to cause the information to be displayed on a terminal device, a portable terminal, or the like used by each person. In yet another example, as for the information of which the notification destination has been determined as a doctor, a nurse, or a receptionist in the clinical department, the notifying function 155 transmits the information to the clinical department system 5 so as to cause the information to be displayed on a terminal device, a portable terminal, or the like used by each person.

In yet another example, as for the information of which the notification destination has been determined as a receptionist in the accounting department, the notifying function 155 transmits the information to the medical accounting system 6 and the hospital staff terminal 10 so as to cause the information to be displayed on a terminal device, a portable terminal, or the like used by each person. In that situation, the information may be displayed when the subject has arrived at the accounting department. Further, when the retention of the subject is completed, a notification indicating which receptionist completed the retention may be provided for the other receptionists. In yet another example, as for the information of which the notification destination has been determined as the subject, the notifying function 155 transmits the information to the subject terminal 7 to notify the subject. In yet another example, as for the treatment execution information of which the notification destination has been determined as the remote image interpretation facility, the notifying function 155 transmits the information to the remote image interpretation facility terminal 8 so as to request an image interpretation doctor to interpret the image data.

In yet another example, as for the information of which the notification destination has been determined as an emergency doctor, a nurse, or a radiologist, the notifying function 155 transmits the information to the electronic medical record system 9 or the hospital staff terminal 10 so as to cause the information to be displayed on a terminal device, a portable terminal, or the like used by each person. Further, for example, by transmitting the information to the electronic medical record system 9, the notifying function 155 is able to input the information to the electronic medical record system 9.

Further, for example, the notifying function 155 is configured to check to see whether the retention information and the treatment execution information provided as the notifications have been acknowledged. In the situation where the retention information or the treatment execution information has not been acknowledged when a predetermined time period (e.g., a number of minutes, a number of hours, or a number of days) has elapsed, the notifying function 155 is capable of notifying the relevant parties of retention information (e.g., a mobile phone number or the address of the subject) and the status of the subject (e.g., having completed emergency treatment, being home, or being hospitalized) and capable of stopping the transmission or the display of the retention information and the treatment execution information. Further, for example, the notifying function 155 is configured to check to see whether the retention of the subject has been completed. In the situation where the retention of the subject has not been completed when a predetermined time period (e.g., a number of minutes, a number of hours, or a number of days) has elapsed, the notifying function 155 is capable of notifying relevant parties of retention information (e.g., a mobile phone number or the address of the subject) and the status of the subject (e.g., having completed emergency treatment, being home, or being hospitalized) and capable of stopping the transmission or the display of the retention information and the treatment execution information.

Next, specific examples of a series of processes related to providing the notification containing the treatment execution information performed by the abovementioned processing functions will be explained by using various situations.

In an example, when CT image data of the chest of a subject has been taken per request of the thoracic surgery department, the analyzing function 152 performs an analyzing process to detect aortic dissections from the image data. After that, when an aortic dissection is detected by the analyzing process, the selecting function 153 selects, from among the plurality of pieces of action information stored in the storage 120, a piece of action information defining the action to notify radiologist A who is in charge of interpreting images, thoracic surgeon B who requested the chest CT images, and thoracic surgeon C who is in charge of surgical operation. In this situation, thoracic surgeon B who requested the chest CT images may be notified of information about thoracic surgeon C who is in charge of surgical operation at the same time.

Subsequently, the determining function 154 generates treatment execution information indicating that the images need to be interpreted urgently and determines radiologist A as a notification destination of the treatment execution information. Further, the determining function 154 generates treatment execution information indicating that a diagnosing process needs to be performed urgently and determines thoracic surgeon B as a notification destination of the treatment execution information. In addition, the determining function 154 generates treatment execution information indicating that a surgical operation needs to be performed urgently and determines thoracic surgeon C as a notification destination of the treatment execution information. Furthermore, in that situation, the determining function 154 may check to see whether or not radiologist A, thoracic surgeon B, and thoracic surgeon C are each at the hospital, and when one or more doctors are absent, the determining function 154 may also determine a substitute candidate doctor (e.g., an emergency surgeon) for the absent doctor as a transmission destination of the treatment execution information. After that, the notifying function 155 transmits the pieces of treatment execution information to radiologist A, thoracic surgeon B, and thoracic surgeon C, or to the substitute candidate doctor thereof.

In yet another example, when CT image data of the chest of a subject has been taken per request of the cardiac surgery department, the analyzing function 152 performs an analyzing process to detect tuberculosis from the image data. After that, when tuberculosis is detected by the analyzing process, the analyzing function 152 further analyzes specimen data of serum collected from the subject and judges whether or not there is a high possibility that tuberculosis may be active tuberculosis. When it is determined that there is a high possibility of being active tuberculosis, the selecting function 153 selects, from among the plurality of pieces of action information stored in the storage 120, a piece of action information defining the action to notify radiologist D and cardiac surgeon E and to also carry out procedures (e.g., contact the public health agency, a method for isolating the subject) according to an infectious disease protocol manual.

Subsequently, the determining function 154 generates treatment execution information indicating that images needs to be interpreted urgently and determines radiologist D as a notification destination of the treatment execution information. Further, the determining function 154 generates treatment execution information indicating that a diagnosing process needs to be performed urgently and indicating the procedure following the infectious disease protocol manual and determines cardiac surgeon E as a notification destination of the treatment execution information. Furthermore, in that situation, for example, when another hospital is designated as a care providing facility by the infectious disease protocol manual, the determining function 154 may generate treatment execution information instructing that an ambulance be arranged and determine a person in charge of the arrangement as a notification destination of the treatment execution information. After that, the notifying function 155 transmits the pieces of treatment execution information to radiologist D and cardiac surgeon E.

In yet another example, when CT image data of the chest of a subject being treated with a cardiotoxic drug has been taken per request of the breast surgery department, the analyzing function 152 performs an analyzing process to detect cardiomegaly from the image data. After that, when cardiomegaly is detected by the analyzing process, the selecting function 153 selects, from among the plurality of pieces of action information stored in the storage 120, a piece of action information defining the action to notify radiologist F and breast surgeon G as well as radiologic technologist H who is to carry out an ultrasound examination.

Subsequently, the determining function 154 generates treatment execution information indicating that the images need to be interpreted urgently and determines radiologist F as a notification destination of the treatment execution information. Further, the determining function 154 generates treatment execution information indicating that a diagnosing process needs to be performed urgently and determines breast surgeon G as a notification destination of the treatment execution information. In addition, the determining function 154 generates treatment execution information instructing that image data of ultrasound images be taken and determines radiologic technologist H as a notification destination of the treatment execution information. After that, the notifying function 155 transmits the pieces of treatment execution information to radiologist F, breast surgeon G and radiologic technologist H.

In this situation, the storage 120 may store therein results of the analyzing processes performed by the analyzing function 152 described above, the action information selected by the selecting function 153, the retention information and the treatment execution information generated by the determining function 154, the determined notification destinations, and/or the like, so that these items can be referenced later.

The processing functions included in the processing circuitry 150 have thus been explained. In this situation, for example, when the processing circuitry 150 is realized by using a processor, the processing functions included in the processing circuitry 150 are each stored in the storage 120 in the form of a computer-executable program. Further, the processing circuitry 150 is configured to realize the processing functions corresponding to the programs, by reading and executing the programs from the storage 120. In other words, the processing circuitry 150 that has read the programs have the processing functions illustrated in FIG. 1.

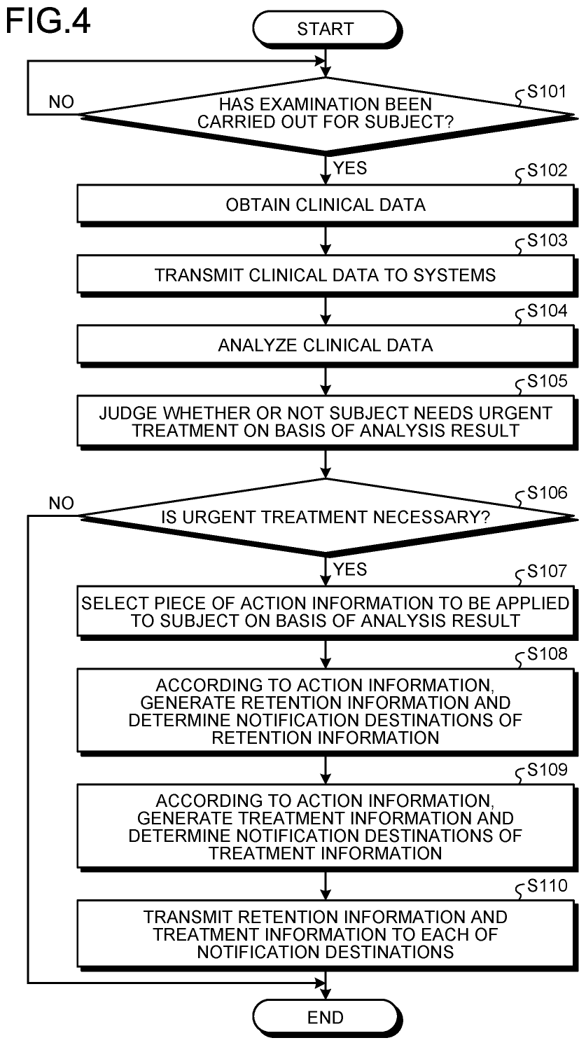

FIG. 4 is a flowchart illustrating a processing procedure of a process performed by the processing circuitry 150 included in the medical information processing apparatus 100 according to the first embodiment.

For example, as illustrated in FIG. 4, in the present embodiment, when an examination is carried out for a subject in the hospital (step S101: Yes), the obtaining function 151 obtains clinical data acquired from the examination (step S102) and transmits the obtained clinical data to the systems (step S103).

Subsequently, the analyzing function 152 performs an analyzing process to analyze the clinical data obtained by the obtaining function 151 (step S104).

Subsequently, on the basis of an analysis result obtained through the analyzing process performed by the analyzing function 152, the selecting function 153 judges whether or not the subject needs urgent treatment (step S105). When it is determined that the urgent treatment is not necessary (step S106: No), the selecting function 153 ends the process without performing the processing procedure thereafter.

On the contrary, when it is determined that the urgent treatment is necessary (step S106: Yes), the selecting function 153 selects a piece of action information to be applied to the subject, from among the plurality of pieces of action information stored in the storage 120, on the basis of the analysis result (step S107).

Subsequently, according to the piece of action information selected by the selecting function 153, the determining function 154 generates retention information for retaining the subject in the hospital and determines one or more notification destinations of the retention information (step S108).

Further, according to the piece of action information selected by the selecting function 153, the determining function 154 generates treatment execution information for notifying that it is necessary to provide urgent treatment for the subject and further determines one or more notification destinations of the treatment execution information (step S109).

After that, the notifying function 155 transmits the retention information and the treatment execution information generated by the determining function 154 to the respective notification destinations thereof (step S110).

In this situation, the processes at steps S101 through S103 described above are realized as a result of, for example, the processing circuitry 150 reading and executing the program corresponding to the obtaining function 151 from the storage 120. Further, the process at step S104 above is realized as a result of, for example, the processing circuitry 150 reading and executing the program corresponding to the analyzing function 152 from the storage 120. Also, the processes at steps S105 through S107 above are realized as a result of, for example, the processing circuitry 150 reading and executing the program corresponding to the selecting function 153 from the storage 120. In addition, the processes at steps S108 and S109 above are realized as a result of, for example, the processing circuitry 150 reading and executing the program corresponding to the determining function 154 from the storage 120. Furthermore, the process at step S110 above is realized as a result of, for example, the processing circuitry 150 reading and executing the program corresponding to the notifying function 155 from the storage 120.

Although the example was explained above in which the processing functions are realized by the single processor, possible embodiments are not limited to this example. It is also acceptable to structure processing circuitry by combining together a plurality of independent processors, so that the processing functions are realized as a result of the processors executing the programs. Further, the processing functions included in the processing circuitry 150 may be realized as being distributed among or integrated together in one or more processing circuits, as appropriate. Further, although the example was explained above in which the single storage (the storage 120) stores therein the programs corresponding to the processing functions, possible embodiments are not limited to this example. It is also acceptable to provide a plurality of storages in a distributed manner, so that the processing circuitry 150 reads a corresponding program from each of the individual storages.

As explained above, in the first embodiment, the storage 120 is configured to store therein the plurality of pieces of action information each defining a medical action corresponding to a state of a disease. Further, the obtaining function 151 is configured to obtain the clinical data related to the subject. The analyzing function 152 is configured to perform the analyzing process to analyze the obtained clinical data. On the basis of the analysis result obtained by the analyzing process, the selecting function 153 is configured to select the piece of action information to be applied to the subject from among the plurality of pieces of action information stored in the storage 120. According to the selected piece of action information, the determining function 154 is configured to generate the retention information for retaining the subject in the hospital and to determine at least one notification destination of the retention information. Further, the notifying function 155 is configured to transmit the retention information to the determined notification destination.

By using this configuration, when the subject needs urgent treatment, it is possible to retain the subject in the hospital. It is therefore possible to provide the urgent treatment while the subject is still in the hospital.

Further, in the first embodiment, according to the selected piece of action information, the determining function 154 is configured to generate the treatment execution information for notifying that it is necessary to provide urgent treatment for the subject and to determine at least one notification destination of the treatment execution information. Further, the notifying function 155 is configured to transmit the treatment execution information to the determined notification destination.

By using this configuration, it is possible to proceed with the procedure for carrying out the urgent treatment, in parallel with retaining the subject in the hospital. It is therefore possible to provide the urgent treatment for the subject more promptly.

As explained above, according to the first embodiment, with respect to the subject who needs urgent treatment, it is possible to prompt the relevant parties to provide the treatment while the subject is still in the hospital. Consequently, according to the present embodiment, it is possible to properly carry out the urgent treatment for the subjects.

Further, because it is possible to carry out the appropriate medical processes for the subject who needs to be treated urgently while he/she is still in the hospital, it is possible to enhance outcomes of the subject and to reduce unnecessary medical costs. Further, in accordance with the status of the hospital and circumstances around the subject, it is possible to provide options optimal for providing the treatment. In addition, by optimizing the notification destinations to be notified of the necessary information as appropriate, it is possible to smoothly proceed with the workflow to provide the treatment.

Further, it is also possible to carry out the first embodiment described above, by modifying a part of the configuration of the medical information processing apparatus 100 as appropriate. Thus, in the following sections, modification examples of the first embodiment will be explained as other embodiments. Differences from the first embodiment will primarily be explained below, while explanations of some of the configurations that are the same as those in the first embodiment will be omitted.

### Second Embodiment

For example, in the first embodiment, the selecting function 153 may be configured to use a trained model generated through machine learning, so as to judge whether or not the subject needs urgent treatment.

For example, the selecting function 153 may be configured to derive a likelihood indicating the possibility that the subject may be hospitalized, by inputting the obtained clinical data to the trained model generated through the machine learning that uses, as training-purpose data, clinical data and information indicating whether or not the subjects in the clinical data were hospitalized. When the derived likelihood is higher than a threshold value, it is determined that urgent treatment is necessary.

In this situation, for example, the trained model may be generated by using any of various types of machine learning methods such as deep learning, a non-linear discriminant analysis, a Support Vector Machine (SVM), a random forest, or a Naive Bayes scheme.

### Third Embodiment

Further, for example, in the first embodiment, when the determining function 154 determines a plurality of notification destinations related to the retention information or the urgent treatment, as the notification destinations of the retention information or the treatment execution information, the notifying function 155 may be configured to simultaneously transmit the retention information or the treatment execution information to the determined notification destinations.

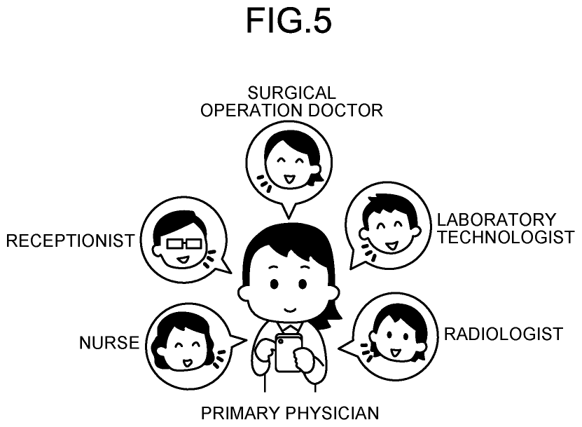

FIG. 5 is a drawing illustrating an example of the simultaneous transmission performed by the notifying function 155 according to a third embodiment.

For example, as illustrated in FIG. 5, when the determining function 154 determines that the primary physician, a surgical operation doctor, a receptionist in the clinical department, a nurse, a laboratory technologist, and a radiologist as notification destinations of the retention information or the treatment execution information, the notifying function 155 is configured to simultaneously transmit the retention information or the treatment execution information to each of the notification destinations.

In the above example, the notifying function 155 is configured to simultaneously transmit the retention information or the treatment execution information to each of the determined notification destinations; however, possible embodiments are not limited to this example. The notifying function 155 may simultaneously transmit only the retention information to each of the notification destinations determined with respect to the retention information or may simultaneously transmit only the treatment execution information to each of the notification destinations determined with respect to the treatment execution information. Further, the notifying function 155 may simultaneously transmit the retention information and the treatment execution information to the notification destinations determined with respect to the retention information and to the notification destinations determined with respect to the treatment execution information.

For example, by using a group organizing function provided by a Social Networking Service (SNS) application, the notifying function 155 may simultaneously provide the plurality of notification destinations with the notifications containing the retention information or the treatment execution information. In another example, by using a simultaneous transmission function provided by an electronic mail (E-mail) application, the notifying function 155 may simultaneously provide the plurality of notification destinations with the notifications containing the retention information or the treatment execution information. In yet another example, by using an application that allows the relevant parties at the notification destinations to share the retention information or the treatment execution information processed by any of the relevant parties, the notifying function 155 may simultaneously provide the plurality of notification destinations with the notifications containing the retention information or the treatment execution information.

In those situations, for example, let us discuss an example in which, when an emergency doctor, a nurse, and a receptionist in the accounting department are determined as notification destinations of the treatment execution information, the retention information or the treatment execution information is to be simultaneously transmitted to each of the notification destinations. On such occasion, in the situation where the subject has already gone away from the emergency doctor and the nurse, the application receives inputs of processed retention information indicating that the subject has already left, from the emergency doctor and the nurse. As a result of receiving the inputs from the emergency doctor and the nurse, the application is able to present the possibility that the subject may show up, to the receptionist in the accounting department.

Further, let us discuss another example in which, when a receptionist in the examination department, a nurse, and a receptionist in the accounting department are determined as notification destinations of the retention information, the application is to simultaneously transmit the retention information to each of the notification destinations. On such occasion, in the situation where the subject has already gone away from the receptionist in the examination department but the receptionist in the examination department was informed of where the subject is headed, the application receives an input of processed retention information indicating where the subject is headed and asking a nurse to look for the subject, from the receptionist in the examination department. As a result of receiving the input from the receptionist in the examination department, the application is able to present the request to carry out the action of looking for the subject, to the nurse.

Further, let us discuss yet another example in which, when an emergency doctor, a nurse, and a receptionist in the accounting department are determined as notification destinations of the retention information or the treatment execution information, the application is to simultaneously transmit the retention information or the treatment execution information to each of the notification destinations. On such occasion, in the situation where the subject has already gone away from the emergency doctor, the application receives, from the emergency doctor, an input of retention information and treatment execution information that have been processed to indicate that the subject is requested to return to the CT room to undergo a CT examination again. As a result of receiving the input from the emergency doctor, the application is able to present the retention information processed to indicate that the subject is requested to return to the CT room when he/she shows up and the treatment execution information processed to indicate the CT re-examination, to the receptionist in the accounting department.

In yet another example, the notifying function 155 may be configured to simultaneously transmit the retention information or the treatment execution information to various types of systems relevant to the notification destinations (e.g., the medical accounting system 6, the remote image interpretation facility terminal 8, the electronic medical record system 9, and the hospital staff terminal 10 or the like) determined by the determining function 154. On such occasion, a controlling function (not illustrated) is able to exercise control, by intermediating communication between the various types of systems, so as to present a terminal in the electronic medical record system 9 and the hospital staff terminal 10 with the transmitted information, the retention information or the treatment execution information that has been processed, a dialog between relevant parties in a group organizing function provided by an SNS application, and the like. On such occasion, the controlling function (not illustrated) may exercise control so as to display, in a list view, information indicating whether the subject is successfully retained, the retention information or the treatment execution information processed by a relevant party, the dialog between the relevant parties, and the like.

Further, the notifying function 155 may detect by whom and when the notification containing the treatment execution information was acknowledged or detect that the treatment execution information has not been acknowledged, so as to save a log indicating the detection result in the storage 120. Further, the notifying function 155 may detect by whom and when the subject was retained or detect that the subject has not been retained, so as to save a log indicating the detection result in the storage 120.

### Fourth Embodiment

Further, for example, in the first embodiment, on the basis of priority levels assigned to the plurality of pieces of action information, the selecting function 153 may be configured to select the piece of action information having the highest priority level.

In that situation, for example, the storage 120 is configured to store therein the plurality of pieces of action information in correspondence with a plurality of conditions such as a state of a disease, the age, and the gender and is also configured to store therein priority levels assigned to the plurality of pieces of action information. Further, for example, on the basis of a result of analyzing the clinical data, the selecting function 153 is configured to select, as candidates, a plurality of pieces of action information having conditions that match the subject from among the plurality of pieces of action information stored in the storage 120 and is configured to further select, from among the selected candidates, the piece of action information having the highest priority level as the piece of action information to be applied to the subject.

### Fifth Embodiment

Further, for example, in the first embodiment, the result of the analyzing process performed by the analyzing function 152 may automatically be entered, as initial information, in the reports created by the radiologist, the pathologist, and the laboratory technologist of the specimen examination.

In that situation, for example, when generating the treatment execution information, the determining function 154 arranges the treatment execution information so as to contain the result of the analyzing process performed by the analyzing function 152. Further, for example, when transmitting the treatment execution information to the notification destinations, the notifying function 155 obtains a template of the report to be created at each of the notification destinations, from among templates of reports that are pre-stored in the storage 120, subsequently enters the analysis result in the template, and transmits the template to the notification destinations together with the treatment execution information.

### Sixth Embodiment

Further, in the first embodiment, the example was explained in which the image data, the pathological data, and the specimen data are used as the clinical data; however, possible forms of the clinical data are not limited to these examples. For instance, it is also acceptable to use audio data having speech of the subject recorded, vital data of the subject, and/or the like.

For example, the obtaining function 151 may be configured to obtain audio data recorded during a consultation for the subject, as the clinical data. The analyzing function 152 may be configured to detect a mental disease or the like suffered by the subject, by analyzing the audio data through an analyzing process such as a natural language analysis. Further, the selecting function 153 may be configured to judge the seriousness of the detected mental disease or the like on the basis of a result of analyzing the audio data. When the determined level of seriousness exceeds a reference level, the selecting function 153 selects a piece of action information to be applied to the subject.

Further, according to the selected piece of action information, the determining function 154 generates retention information and treatment execution information. The notifying function 155 transmits the plurality of pieces of information to the respective notification destinations thereof. For example, the retention information is generated to instruct that the subject be retained in the hospital and that the subject's mental state be calmed down, so as to be transmitted to a nurse in the clinical department. Further, the treatment execution information is generated to instruct that the subject take a tranquilizing drug, so as to be transmitted to a nurse in the clinical department and to the primary physician.

In another example, the obtaining function 151 may be configured to obtain, as the clinical data, vital data acquired by a smartphone, a wearable terminal, or the like owned by the subject. The analyzing function 152 detects a disease suffered by the subject, by analyzing the vital data through an analyzing process to analyze vital values. Further, on the basis of a result of analyzing the vital data, the selecting function 153 judges whether or not the subject needs urgent treatment. When it is determined that urgent treatment is necessary, the selecting function 153 selects a piece of action information to be applied to the subject.

Further, according to the selected piece of action information, the determining function 154 generates retention information and treatment execution information. The notifying function 155 transmits the plurality of pieces of information to the respective notification destinations thereof. For example, when the subject is using a wheelchair that can be controlled and operated with a smartphone, the retention information is generated to instruct that the wheelchair be directed to the inside of the hospital or to the clinical department of the primary physician, so as to be transmitted to the smartphone of the subject. Further, the treatment execution information is generated to notify that the subject is headed for the clinical department on a wheelchair and that the subject needs a diagnosing process urgently, so as to be transmitted to the primary physician.

### Seventh Embodiment

Further, in the first embodiment, the example was explained in which, according to the piece of action information selected by the selecting function 153, the determining function 154 is configured to generate the retention information for retaining, in the hospital, the subject determined to be requiring urgent treatment and is configured to determine at least one notification destination of the retention information; however, possible modes of the notification destinations to be determined are not limited to this example. For example, the determining function 154 may be configured to compare the time at which a relevant party at the notification destination of the retention information became aware of the retention information, with a time at which it was possible to recognize the status of the subject (e.g., a time at which imaging was performed; a time at which an examination result was registered; a time at which medical care was completed; or a time at which the accounting process was completed) and which can be obtained from any of the various types of systems such as the electronic medical record system 9, so as to change the retention information or the transmission destinations of the retention information. As a result of the hospital staff inputting the retention information to the hospital staff terminal 10 or the like, the hospital staff terminal 10 or the like obtains the time at which the staff becomes aware of the retention information.

For example, if the time at which the relevant party at the notification destination of the retention information became aware of the retention information is later than the time at which the accounting process is completed, the relevant party may receive, as the retention information, a notification containing contact information (e.g., a mobile phone number and/or the address, etc.) to be used for contacting the subject.

On the contrary, for example, if the time at which the relevant party at the notification destination of the retention information became aware of the retention information is earlier than the time at which medical care was completed in the emergency department, the emergency doctor may receive a notification containing the retention information and treatment execution information. In other words, the notification destinations of the retention information may vary depending on whether the time (timing) at (with) which the retention information was noticed was before the specific event or after the specific event. The determining function 154 may be configured to obtain the time at which the retention information was noticed (the retention information was obtained) and to determine at least one notification destination of the retention information in accordance with whether the time at which the retention information was obtained was before the specific event or after the specific event.

### Eighth Embodiment

Further, in the first embodiment, the example was explained in which the image data of the subject is taken in the hospital; however, possible embodiments are not limited to this example. The techniques disclosed in the present disclosure are also applicable to the situation where, for example, the image data of the subject is taken at an imaging center dedicated for imaging.

In that situation, for example, the obtaining function 151 is configured to obtain, as the clinical data, the image data of the subject from an image storing device installed in the image center. Further, the analyzing function 152 is configured to perform an analyzing process to analyze the image data. On the basis of an analysis result obtained from the analysis process, the selecting function 153 is configured to judge whether or not the subject needs urgent treatment. When it is determined that urgent treatment is necessary, the selecting function 153 is configured to select a piece of action information to be applied to the subject. After that, according to the selected piece of action information, the determining function 154 is configured to generate retention information and treatment execution information. The notifying function 155 is configured to transmit the plurality of pieces of information to the respective notification destinations thereof.

For example, the determining function 154 generates retention information notifying that the subject is to be transported in an ambulance, so that the notifying function 155 transmits the retention information to the subject himself/herself. Further, for example, the determining function 154 generates treatment execution information instructing that an ambulance be arranged, so that the notifying function 155 transmits the treatment execution information to the physician of the subject and to a person in charge of the arrangement at the hospital. Further, for example, the determining function 154 generates treatment execution information notifying that the subject is to be transported in an ambulance, so that the notifying function 155 transmits the treatment execution information to the insurance company of an insurance owned by the subject's family or by the subject. The retention information and the treatment execution information may sequentially be transmitted in the sequential order stated above or may simultaneously be transmitted.

Further, for example, when transmitting the treatment execution information to the insurance company, the determining function 154 may change the content of the treatment execution information in accordance with the content or the type of the insurance owned by the subject. Further, for example, to transmit the treatment execution information to the primary physician of the subject, the determining function 154 may generate from time to time treatment execution information indicating movements (the positions, the state, etc.) of the subject since the retention information was transmitted to the subject.

In another example, the determining function 154 may generate information instructing that the subject wait at home until being contacted by the primary physician as retention information to be transmitted to the subject and may generate information instructing the primary physician to contact the subject as treatment execution information to be transmitted to the primary physician. In yet another example, the determining function 154 may generate, as retention information to be transmitted to the subject, information instructing that the subject undergo a remote diagnosis/treatment session or information notifying that the subject is to be transported to the hospital.

By using these configurations, it is possible to quickly transport the subject to the hospitalization facility or the treatment facility, even when the image data of the subject is taken at the image center. It is therefore possible to promptly interfere with the subject even when the disease requires urgent attention.

Some of the embodiments related to the medical information processing apparatus have thus been explained; however, possible embodiments of the techniques disclosed in the present disclosure are not limited to these examples.

For instance, in the embodiments above, the medical information processing apparatus 100 includes the analyzing function 152; however, possible embodiments are not limited to this example. For instance, the clinical data acquiring device 1 may include an analyzing function. In that situation, the analyzing function of the clinical data acquiring device 1 is configured to perform the analyzing process to analyze the clinical data acquired from the examination and to transmit an analysis result thereof to the medical information processing apparatus 100.

In yet another example, the configuration of the medical information processing apparatus 100 described in any of the embodiments above may be realized by using a cloud server. In that situation, for example, every time an examination for a subject is carried out in the hospital, the clinical data acquiring device 1 is configured to transmit the clinical data obtained from the examination to the cloud server. After that, by performing the same processes as those performed by the obtaining function 151, the analyzing function 152, the selecting function 153, the determining function 154, and the notifying function 155 included in the medical information processing apparatus 100 described above, the cloud server is configured to transmit the retention information and the treatment execution information to the respective notification destinations thereof.

The various types of data handled in the present disclosure are, typically, digital data.

Further, in the embodiments above, the example was explained in which the obtaining unit, the analyzing unit, the selecting unit, the determining unit, and the notifying unit of the present disclosure are realized, respectively, by the obtaining function 151, the analyzing function 152, the selecting function 153, the determining function 154, and the notifying function 155 of the processing circuitry 150; however, possible embodiments are not limited to this example. For instance, instead of realizing the obtaining unit, the analyzing unit, the selecting unit, the determining unit, and the notifying unit of the present disclosure with the obtaining function 151, the analyzing function 152, the selecting function 153, the determining function 154, and the notifying function 155 described in the embodiments, it is also acceptable to realize these functions by using only hardware, only software, or a combination of hardware and software.

Further, the term “processor” used in the above explanations denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). When the processor is a CPU, for example, the processor is configured to realize the functions by reading and executing the programs saved in the storage. Alternatively, when the processor is an ASIC, the functions are directly incorporated, as a logic circuit, into the circuit of the processor, instead of the programs being saved in the storage. Further, the processors according to the present embodiments do not each necessarily have to be structured as a single circuit. It is also acceptable to structure one processor by combining together a plurality of independent circuits, so as to realize the functions thereof. Further, two or more of the constituent elements illustrated in FIG. 1 may be integrated in a single processor so as to realize the functions thereof.

In this situation, the programs executed by the one or more processors are provided as being incorporated, in advance, in a Read Only Memory (ROM), a storage, or the like. The programs may be provided as being recorded in a computer-readable medium such as a Compact Disk Read-Only Memory (CD-ROM), a Flexible Disk (FD), a Compact Disk Recordable (CD-R), a Digital Versatile Disk (DVD), or the like, in a file in a format that is installable or executable by these devices. Further, the programs may be stored in a computer connected to a network such as the Internet so as to be provided or distributed as being downloaded via the network. For example, the programs are structured with modules including the functional units described above. In the actual hardware, as a result of a CPU reading and executing the programs from a storage medium such as a ROM, the modules are loaded into a main storage device and generated in the main storage device.

According to at least one aspect of the embodiments described above, it is possible to properly provide the urgent treatment for the subjects.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising:
a storage configured to store a plurality of pieces of action information each defining a medical action corresponding to a state of a disease; and
processing circuitry configured to:
obtain clinical data related to a subject;
perform an analyzing process to analyze the obtained clinical data;
select a piece of action information to be applied to the subject from among the plurality of pieces of action information stored in the storage, on a basis of an analysis result obtained from the analyzing process;
generate retention information for retaining the subject in a medical facility and determine at least one notification destination of the retention information, according to the selected piece of action information; and
transmit the retention information to the determined notification destination.

2. The medical information processing apparatus according to claim 1, wherein
the processing circuitry is further configured to:
generate treatment execution information for notifying that it is necessary to provide urgent treatment for the subject and determine at least one notification destination of the treatment execution information, according to the selected piece of action information; and
transmit the treatment execution information to the determined notification destination.

3. The medical information processing apparatus according to claim 1, wherein
the processing circuitry is further configured to judge whether or not the subject needs urgent treatment on a basis of the analysis result, and, when it is determined that the urgent treatment is necessary, select the piece of action information to be applied to the subject.

4. The medical information processing apparatus according to claim 3, wherein
the processing circuitry is further configured to derive a likelihood indicating a possibility that the subject may be hospitalized, by inputting the obtained clinical data to a trained model generated through machine learning that uses, as training-purpose data, clinical data and information indicating whether or not subjects in the clinical data were hospitalized, and, when the likelihood is higher than a threshold value, determine that the urgent treatment is necessary.

5. The medical information processing apparatus according to claim 1, wherein
the processing circuitry is further configured to generate, as the retention information, information instructing a healthcare worker to retain the subject in the medical facility.

6. The medical information processing apparatus according to claim 2, wherein
the processing circuitry is further configured to generate, as the treatment execution information, information instructing a healthcare worker about an item or a procedure of the urgent treatment.

7. The medical information processing apparatus according to claim 2, wherein
the processing circuitry is further configured to generate, as the treatment execution information, information indicating a degree of urgency of the urgent treatment for the subject.

8. The medical information processing apparatus according to claim 1, wherein
the processing circuitry is further configured to obtain a current position of the subject, and determine the notification destination of the retention information in accordance with the current position.

9. The medical information processing apparatus according to claim 2, wherein
the processing circuitry is further configured to determine the notification destination of one of the retention information and the treatment execution information, in accordance with a requester that requested the clinical data.

10. The medical information processing apparatus according to claim 2, wherein
the processing circuitry is further configured to:
determine, as the notification destination of one of the retention information and the treatment execution information, a plurality of notification destinations relevant to one of the retention information and the urgent treatment; and
simultaneously transmit the one of the retention information and the treatment execution information to the determined plurality of notification destinations.

11. The medical information processing apparatus according to claim 2, wherein
the processing circuitry is further configured to obtain one of the retention information and the treatment execution information that has been processed at one of a plurality of notification destinations, and provide the obtained information again as a notification.

12. The medical information processing apparatus according to claim 10, wherein
the processing circuitry is further configured to:
simultaneously transmit one of the retention information and the treatment execution information to systems relevant to the determined plurality of notification destinations; and
exercise control by intermediating communication between the systems, so as to present the plurality of notification destinations with one of the following: the retention information, the treatment execution information, the retention information that has been processed at one of the notification destinations, the treatment execution information that has been processed at one of the notification destinations, and a dialogue between relevant parties.

13. The medical information processing apparatus according to claim 2, wherein
the processing circuitry is further configured to determine a sequential order in which a plurality of notification destinations are to be notified.

14. The medical information processing apparatus according to claim 13, wherein
the processing circuitry is further configured to receive, from a healthcare worker at any of the notification destinations, an instruction indicating that transmission thereafter be cancelled or an instruction indicating that a sequential order of transmission be changed.

15. The medical information processing apparatus according to claim 1, wherein
the processing circuitry is further configured to receive, from a healthcare worker at the notification destination, a designation of an additional notification destination, and notify the additional notification destination again.

16. The medical information processing apparatus according to claim 1, wherein
the processing circuitry is further configured to acquire a time at which the retention information has been obtained, and determine the at least one notification destination of the retention information, in accordance with whether the time at which the retention information has been obtained is before a specific event or after the specific event.

17. The medical information processing apparatus according to claim 1, wherein
the processing circuitry is further configured to obtain subject information indicating up to which process in a workflow has been completed, the workflow starting with the subject taking the examination and ending with a specific treatment method for the subject being determined, and determine the at least one notification destination of the retention information on a basis of the subject information.

18. The medical information processing apparatus according to claim 1, wherein
the processing circuitry is further configured to compare a time at which a relevant party at the notification destination of the retention information became aware of the retention information with a time at which it was possible to recognize a status of the subject, and change one of the retention information and a transmission destination of the retention information.

19. A medical information processing apparatus comprising:
the processing circuitry configured to:
obtain clinical data related to a subject;
perform an analyzing process to analyze the obtained clinical data;
generate retention information for retaining the subject in a medical facility and determine at least one notification destination of the retention information, on a basis of an analysis result obtained from the analyzing process; and
transmit the retention information to the determined notification destination.

20. A medical information processing method comprising:
storing a plurality of pieces of action information each defining a medical action corresponding to a state of a disease;
obtaining clinical data related to a subject;
performing an analyzing process to analyze the obtained clinical data;
selecting a piece of action information to be applied to the subject from among the stored plurality of pieces of action information, on a basis of an analysis result obtained from the analyzing process;
generating retention information for retaining the subject in a medical facility and determining at least one notification destination of the retention information, according to the selected piece of action information; and
transmitting the retention information to the determined notification destination.

21. A medical information processing method comprising:
obtaining clinical data related to a subject;
performing an analyzing process to analyze the obtained clinical data;
generating retention information for retaining the subject in a medical facility and determining at least one notification destination of the retention information, on a basis of an analysis result obtained from the analyzing process; and
transmitting the retention information to the determined notification destination.

22. A non-transitory computer-readable medium having plural computer-executable instructions recorded therein, the instructions causing a computer to execute:
storing a plurality of pieces of action information each defining a medical action corresponding to a state of a disease;
obtaining clinical data related to a subject;
performing an analyzing process to analyze the obtained clinical data;
selecting a piece of action information to be applied to the subject from among the stored plurality of pieces of action information, on a basis of an analysis result obtained from the analyzing process;
generating retention information for retaining the subject in a medical facility and determining at least one notification destination of the retention information, according to the selected piece of action information; and
transmitting the retention information to the determined notification destination.

23. A non-transitory computer-readable medium having plural computer-executable instructions recorded therein, the instructions causing a computer to execute:
obtaining clinical data related to a subject;
performing an analyzing process to analyze the obtained clinical data;
generating retention information for retaining the subject in a medical facility and determining at least one notification destination of the retention information, on a basis of an analysis result obtained from the analyzing process; and
transmitting the retention information to the determined notification destination.
